(19) United States
(12) Patent Application Publication
(10) Pub. No.: US 2021/0148888 A1
(43) Pub. Date: May 20, 2021

(54) Accurate Sensing of Physiological Substance in Blood

(71) Applicant: Joanneum Research Forschungsgesellschaft MBH, Graz (AT)

(72) Inventors: Bruna Pekec, Graz (AT); Martin Hajnsek, Graz (AT)

(21) Appl. No.: 16/637,296

(22) PCT Filed: Aug. 9, 2018
(86) PCT No.: PCT/EP2018/071662

§ 371 Date: Feb. 7, 2020

(30) Foreign Application Priority Data
Aug. 9, 2017 (DE) 10 2017 118 147.4

(51) Int. Cl. G01N 33/49; G01N 27/333

(52) U.S. Cl. CPC G01N 33/491; G01N 27/333; G01N 33/492

(57) ABSTRACT

A sensor arrangement for detecting information indicative of a physiological substance in blood of a physiological subject, wherein the sensor arrangement has a first sensor structure configured for electrochemically sensing the physiological substance in at least part of the blood, wherein detecting the physiological substance is influenceable by hemolysis of the blood, a second sensor structure configured for electrochemically sensing a parameter in at least part of the blood, which parameter is indicative of hemolysis of the blood, and a plasma separation unit configured for separating plasma from the blood and arranged so that the separated plasma is supplied to the first sensor structure and the second sensor structure, whereas a rest of the blood is kept apart from the first sensor structure and the second sensor structure.

### CROSS-REFERENCE TO RELATED APPLICATION

This application is the U.S. national phase of International Application No. PCT/EP2018/071662 filed 9 Aug. 2018 which designated the U.S. and claims priority to German Patent Application No. 10 2017 118 147.4 filed 9 Aug. 2017, the entire contents of each of which are hereby incorporated herein by reference.

### TECHNICAL FIELD

Embodiments of the invention relate to a sensor arrangement. Moreover, embodiments of the invention relate to a monitoring system. Beyond this, embodiments of the invention relate to a detection method.

### TECHNOLOGICAL BACKGROUND

A potassium test is used to measure the amount of potassium in a patient's blood. Potassium is an electrolyte that is essential for proper muscle and nerve function. Electrolytes become ions when they are in a solution, and they conduct electricity. Human cells and organs require electrolytes to function normally. Even minor increases or decreases in the amount of potassium in a patient's blood can result in serious health problems.

A potassium test can be performed as a blood test. A blood sample drawn by a patient's healthcare provider will be sent to a laboratory for analysis, and a doctor will review the results.

However, conventional potassium tests and other blood tests frequently suffer from poor accuracy and the need to involve medical experts.

### SUMMARY

There may be a need to provide a blood test with high accuracy and low effort.

A sensor arrangement, a monitoring system, and a detecting method are provided.

According to an exemplary embodiment of the invention, a sensor arrangement for detecting information indicative of a physiological substance (for instance detecting an amount or a concentration of the physiological substance) in blood of a physiological subject is provided, wherein the sensor arrangement comprises a first sensor structure configured for sensing the physiological substance in at least part of the blood, wherein detecting the physiological substance is influenceable by hemolysis of the blood, and a second sensor structure configured for sensing a parameter in at least part of the blood, which parameter is indicative of hemolysis of the blood.

According to another exemplary embodiment of the invention, a method (in particular carried out in vitro) of detecting information indicative of a physiological substance (for instance detecting an amount or a concentration of the physiological substance) in blood of a physiological subject is provided, wherein the method comprises sensing the physiological substance in at least part of the blood, wherein detecting the physiological substance is influenceable by hemolysis of the blood, and sensing a parameter in at least part of the blood, which parameter is indicative of hemolysis of the blood.

According to still another exemplary embodiment of the invention, a monitoring system is provided which comprises a sensor arrangement having the above mentioned properties, and an evaluation unit configured for evaluating sensed information (such as an evaluation of the amount or concentration of the physiological substance, and/or an evaluation of the presence, absence or strength of hemolysis, and/or an evaluation of a degree of impact of detected hemolysis on the accuracy or inaccuracy of a detected amount or concentration of the physiological substance).

### OVERVIEW OF EMBODIMENTS

The term “physiological subject” or biological object may particularly denote any human being, and any other animal or organism.

The term “physiological substance” may particularly denote any substance which occurs naturally within a physiological subject and is therefore related to the physiology of a living organism, for instance the metabolism, etc. Such a physiological substance may include a biochemically active molecule (such as potassium, glucose), a hormone, a protein, an enzyme, etc.

The term “hemolysis” may particularly denote the rupturing or lysis of red blood cells (which may also be denoted as erythrocytes) and the release of their contents (such as cytoplasm) into surrounding fluid (in particular blood plasma). Hemolysis may occur in vivo and/or in vitro (i.e. inside and/or outside the body).

The term “detecting the physiological substance is influenceable by hemolysis of the blood” may particularly denote that the chemical and/or physical properties of the physiological substance to be detected and/or its detectability may depend on or may be influenced by the presence or absence and/or the amount of hemolysis of the blood in which the physiological substance to be detected is present.

The term “plasma separation” may particularly denote a process of separating blood (in particular whole blood) into blood plasma and blood cells (in particular only blood cells having a size larger than a predetermined threshold value). In an embodiment, larger blood cells (such as erythrocytes) may be prevented from passing a plasma separation unit, whereas smaller blood cells (such as thrombocytes) may pass the plasma separation unit. In another embodiment, all blood cells (such as erythrocytes and thrombocytes) may be prevented from passing the plasma separation unit. When the plasma separation unit is embodied as a permeable membrane, the molecular weight cut off (MWCO) of such a membrane defines which particles can pass through the membrane, and which not. The molecular weight cut off is correlated with pore sizes of pores in the membrane. Subsequently, selectively the separated plasma may be supplied to a desired location or destination of a sensor arrangement for actual detection.

The term “test strip” may particularly denote a band or piece or strip (for instance of plastic, paper, ceramic, metal and/or other material) used for biological testing. Test strips may be handy mobile laboratories for the semiquantitative detection of ions, organic substances, inorganic substances, etc.

A gist of an exemplary embodiment of the invention is that a first sensor structure sensing a hemolysis-sensitive physiological substance in blood is combined with a second sensor structure sensing hemolysis in the same blood sample. The result of the hemolysis detection can then be used for interpreting the results of the detection of the hemolysis-sensitive physiological substance which can be disturbed by hemolysis. Since the event of hemolysis has the consequence that the drawn blood sample is no longer characteristic for the properties of the blood in the physiological subject (such as a human being), considering hemolysis for determining the physiological substance increases accuracy and reliability of the result. Thus, hemolysis related artefacts in the measurement of the hemolysis-sensitive physiological substance may be efficiently suppressed by considering the reference measurement in form of the hemolysis detection. As a result, the detection of the hemolysis sensitive physiological substance (for instance potassium or an enzyme) is more accurate and more reliable. In addition, the first sensor structure and the second sensor structure may both be configured for electrochemically sensing the physiological substance and the parameter, respectively, so that both sensor structures may share or make use of common equipment, such as a common potentiostat, for the electrochemical sensing. As a result, space consumption may be reduced and the sensor arrangement may thus be provided in a more compact manner.

A further gist of an exemplary embodiment of the invention is that a measurement of a physiological substance and/or of a (physiological) parameter is not carried out on the basis of a whole blood sample, but is carried out selectively on the basis of separated blood plasma only. For that purpose, a plasma separation can be executed before measuring the physiological substance and/or the (physiological) parameter in the separated plasma being substantially free of blood cells. By such a passive plasma separation, a measurement of a physiological substance and/or of a (physiological) parameter in blood can become more accurate, since the measurement of several physiological parameters such as the potassium concentration and/or a parameter indicative of hemolysis can be carried out in a more precise way on the basis of blood plasma rather than on the basis of whole blood.

In the following, further exemplary embodiments of the sensor arrangement, the monitoring system, and the method will be explained.

As mentioned above, the evaluation unit may be configured for evaluating sensed information. In an embodiment, the evaluation unit may determine the information indicative of the physiological substance by evaluating the sensed data captured by the first sensor structure (relating to the sensing of the physiological substance) in view of the sensor data captured by the second sensor structure (relating to the sensing of hemolysis). This may involve a correction of the sensed information concerning the physiological substance in view of the sensed hemolysis. In an embodiment, the evaluation unit may determine the information indicative of the physiological substance and/or of the (physiological) parameter by evaluating the sensed data captured by the sensor structure (relating to the sensing of the physiological substance and/or of the (physiological) parameter) selectively based on the separated plasma portion of the blood. It is also possible that an evaluation unit carries out more than one of the mentioned tasks.

In an embodiment, the first sensor structure is configured so that detecting the physiological substance is hemolysis-sensitive. In other words, the result of the detection of the physiological substance by the first sensor structure may be influenced by hemolysis of the blood sample under investigation. Thus, additional detection of hemolysis in the same blood sample may render the result of the detection of the physiological substance more reliable.

In an embodiment, the first sensor structure is configured for sensing potassium as the physiological substance. In other words, the first sensor structure may be configured for potassium sensing. Thus, the first sensor structure may determine the potassium ion (i.e. K±) concentration in the blood sample, or more preferably in the previously separated plasma of the blood sample only. Since the determination of potassium ion concentration in blood depends sensitively on the fact as to whether the blood sample has suffered hemolysis, an additional determination of a hemolysis related parameter in the blood sample may be advantageous for improving accuracy of the potassium determination.

In an embodiment, the first sensor structure is configured for electrochemically sensing the physiological substance. Electrochemistry refers to the relationship between electricity, as a measurable and quantitative phenomenon, and identifiable chemical change or properties. In an embodiment, the electricity caused by the physiological substance (such as potassium ions) can be considered as an outcome of a particular chemical property of the blood sample under investigation, i.e. its amount or concentration of the physiological substance. Reactions used during an electrochemical sensing of a physiological substance may involve electric charges moving between electrodes and an electrolyte or ionic species in a solution.

More specifically, the first sensor structure may comprise a reference electrode (in particular a silver/silver chloride (Ag—AgCl) reference electrode) and an ion-sensitive electrode for detecting the physiological substance electrochemically. The reference electrode stays or is kept at a constant electric potential while the ion-sensitive electrode changes its electric potential with the potassium concentration in the sample. The potential difference between the two electrodes may be measured as an electric voltage. This potential difference is indicative of the potassium concentration in the blood. A corresponding first sensor structure with planar electrodes may be manufactured with low effort, for instance by screen printing. Moreover, a corresponding sensor arrangement may be manufactured in a compact flat configuration.

Thus, the first sensor structure (in particular configured for sensing potassium concentration) may be embodied as a potentiometric sensor, i.e. a sensor detecting an electric voltage. As a result, the first sensor structure may be embodied with two electrodes.

In an embodiment, the second sensor structure is configured for electrochemically sensing hemolysis (in particular by electrochemically detecting iron). For instance, hemolysis in a blood sample may be detected by using an electrode to measure the reduction of hydrogen peroxide concentration (and/or another oxidizing agent) when hemoglobin in plasma is oxidized or to directly measure hemoglobin, for instance by determining the iron in the hemoglobin. Thus, also the second sensor structure may use an electrochemical measurement principle for determining information about hemolysis.

In an embodiment, the second sensor structure is configured for electrochemically sensing hemolysis by directly measuring hemoglobin, for instance by determining the iron in the hemoglobin. This approach might be advantageous over an indirect measurement of hemoglobin, such as by measuring hydrogen peroxide (and/or another oxidizing agent) when hemoglobin in plasma is oxidized. Typically, the hydrogen peroxide measured in an indirect measurement of hemoglobin originates from glucose. Thus, an indirect measurement of hemoglobin by measuring hydrogen peroxide typically requires the additional measurement of the glucose level. By directly measuring hemoglobin, in particular by a direct electrochemical sensing of hemoglobin, the additional measurement (as well as the equipment required for doing so) of the glucose level may be omitted which may improve the efficiency and the accuracy of the determination of the hemolysis-indicative parameter as well as the compactness of the sensor arrangement.

More specifically, the second sensor structure may comprise a reference electrode, a working electrode (which may also be denoted as a detection electrode) and a counter electrode. The working electrode may be kept at a constant electric working potential that is used to carry out a redox reaction with the iron ions in hemoglobin. The measured electric current between working electrode and counter electrode is indicative for the concentration of free hemoglobin in the sample. The concentration of free hemoglobin in the sample, in turn, is correlated with the amount of hemolysis of the blood. A corresponding second sensor structure with planar electrodes may be manufactured with low effort, for instance by screen printing. Moreover, a corresponding sensor arrangement may be manufactured in a compact flat configuration. This particularly holds when planar electrodes of the first sensor structure and of the second sensor structures are all formed coplanar, i.e. in a common plane.

Thus, the second sensor structure (in particular configured for sensing hemolysis) may be embodied as an amperometric sensor, i.e. a sensor detecting an electric current. As a result, the second sensor structure may be embodied with three electrodes.

In an embodiment, the second sensor structure is configured for optically sensing hemolysis (for instance by detecting the red color of the blood sample or part thereof, preferably only the plasma part of the blood sample). For instance, hemolysis may be assessed by an optical measurement of hemoglobin released into the supernatant. Thus, as a complementary addition for increased accuracy or as an alternative to the previously described electrochemical detection of hemolysis, also an optical detection of hemolysis can be implemented in the sensor arrangement. For that purpose, electromagnetic radiation (for instance light in the visible range) may be detected after propagation through the blood sample or after reflection by the blood sample. An electromagnetic radiation source may be provided for irradiating the blood sample with the electromagnetic radiation. By taking this measure, reflectivity, absorption, transmissivity, and/or another optical parameter of the blood sample may be measured which is characteristically influenced by hemolysis.

In an embodiment, the first sensor structure and the second sensor structure are arranged on and/or integrated in a common test strip. By taking this measure, the detection of the physiological substance and the hemolysis detection may be carried out spatially very close together. Therefore, the measurements in terms of the physiological substance and the potential hemolysis are directly comparable with high accuracy. At the same time, a very small amount of blood may be sufficient for carrying out both measurements. A corresponding sensor arrangement may also be configured as a simply manufacturable test strip which a patient can use at home.

In an embodiment, the sensor arrangement is configured as a disposable test strip operable by a patient, even without medical personnel. Due to a hemolysis detection capability integrated on a test strip, undesired hemolysis of a blood sample by improper operation by a patient (who is in many cases a medical laymen) will advantageously not result in a deterioration of the detection of the physiological substance, even when the latter is hemolysis-sensitive. Due to the simple sheet like configuration of the individual constituents of the sensor arrangement and its cost efficient manufacturability by simple processes such as screen printing, the sensor arrangement is also suitable to be used as a single-use or disposable device. A corresponding test strip may be easily operated even by patients without professional medical skills.

In an embodiment, the second sensor structure is configured for sensing hemolysis qualitatively or quantitatively. In other words, the sensor structure may be configured for sensing one of the group consisting of the presence or absence of hemolysis in the blood, and an amount of hemolysis in the blood. A qualitative information about hemolysis may be a simple yes or no result. A quantitative information about hemolysis may be indicative of a value indicating the strength or level of hemolysis. The qualitative or quantitative hemolysis information may be used for interpreting or correcting the results of the hemolysis sensitive detection of the physiological substance.

In an embodiment, the sensor arrangement comprises a plasma separation unit configured for separating plasma from or of the blood sample and arranged so that selectively the separated plasma (optionally still comprising small blood cells, but being free of larger blood cells) is supplied to the first sensor structure and/or the second sensor structure. Thus, the plasma separation unit may be provided upstream of the first sensor structure and/or the second sensor structure. By such a plasma separation unit, blood plasma (i.e. water, proteins, ions, optionally small cells, etc.) may be separated from cells of the whole blood. Only the plasma may then be supplied to the sensor structure(s), whereas a rest of the blood (in particular cells, more particularly large cells) may be kept apart from the sensor structure(s). Determination of many physiological substances as well as of hemolysis may be carried out with significantly better precision on the basis of plasma than on the basis of whole blood.

In an embodiment, the plasma separation unit may comprise or consist of a filter filtering the blood sample. Such a filter may be embodied as a permeable membrane being permeable only for a part of the constituents of the blood (i.e. may be permeable for the plasma and impermeable for the larger cells of the blood). More specifically, a permeable membrane may be provided as filter having pores which has a smaller diameter on a main surface facing the sensor structure compared to an opposing other main surface facing a user supplying blood to the sensor arrangement. By taking this measure, the active surface of the filter on a side facing the blood sample provided by the user may be enlarged, so that undesired clogging of the filter at this exterior side can be efficiently prevented. For example, the pores may have a size at the surface facing the user's finger larger than essentially 1 μm, particularly in the range between essentially 1 μm and essentially 10 μm. The pores may have a size at the opposing surface facing the sensor structure(s) smaller than essentially 1 μm, particularly in the range between essentially 0.2 μm and essentially 1 μm. Such a membrane may be prepared by extrusion from polysulfone, polyethersulfone or a blend of polyethersulfone and polyvinylpyrrolidone, or on the basis of another polymer. Other possible membrane materials may include inorganic materials like ceramic, glass, stainless steel or derivatized carbon. Undesired clogging of the membrane by large blood cells may be prevented due to the relatively large pore size on the side of the membrane at which the raw blood drop is supplied from the patient. The smaller pores on the opposing other side allow for an efficient transfer of the filtered plasma to the fluidic channel and from there to the one or more sensor structures. However, as an alternative to such a membrane with an anisotropic or inhomogeneous pore structure, it is also possible that a membrane with homogeneous or isotropic pore size over its thickness extension may be used.

In an embodiment, the sensor arrangement comprises a fluidic channel structure, in particular a microfluidic channel structure (i.e. a channel structure having dimensions in the order of micrometers), configured for guiding at least part of the blood (in particular the separated plasma output from the plasma separation unit) to the first sensor structure and the second sensor structure. In such a fluidic channel structure, a capillary or other fluidic conduit may be delimited to precisely define a trajectory along which the blood or part thereof may flow in a defined way (in particular straight to the sensor structure(s)).

Preferably, the guidance of the blood or parts thereof (in particular plasma) along the fluidic channel may be powered or driven by capillary action. More specifically, the sensor arrangement may comprise a sheet with a microfluidic channel configured for guiding the blood or part thereof (in particular the plasma), i.e. the sample fluid, to the sensor structure by capillary action. Capillary action may be denoted as the ability of the blood or part thereof to flow in narrow spaces without the assistance of, or even in opposition to, external forces like gravity. The effect occurs because of intermolecular forces between the blood or part thereof and surrounding solid surfaces delimiting the fluidic channel. If the diameter of the fluidic channel is sufficiently small, then the combination of surface tension (which is caused by cohesion within the blood or part thereof) and adhesive forces between the blood or part thereof and the wall of the fluidic structure act to propel the blood or part thereof. When a microfluidic channel is implemented which generates a capillary force for moving the blood or part thereof to the sensor structure(s), a fluidic pump or another driving unit for driving the sample fluid becomes dispensable. This allows manufacturing the sensor arrangement with low effort and of a test strip type.

As an alternative to a sheet with a fluidic channel, it is also possible to provide a sheet with pronounced liquid absorbing capability, such as a piece of blotting paper.

In an embodiment, at least one of the group consisting of the first sensor structure, the second sensor structure, the plasma separation unit, the fluidic channel structure, and the sensor arrangement as a whole, is configured as a flat sheet. In particular, all of the mentioned constituents of the sensor arrangement may be formed on the basis of a respective flat sheet or sheet portion. More specifically, the sensor arrangement as a whole may be configured as flat sheet and hence highly compact. The first sensor structure and the second sensor structure may be formed on the basis of flat electrodes structures, preferably all located within a common plane. The plasma separation unit may be a flat filter sheet defined by a porous membrane. The fluidic channel structure may be defined by a recess in a sheet, which recess may be covered by a further sheet so as to fully circumferentially delimit a capillary. When these constituents, which may be all configured as flat sheet structures, are stacked, a sheet type sensor arrangement may be obtained which is configured as a test strip (see FIG. 3 and FIG. 4).

In an embodiment, the sensor arrangement comprises a semiconductor chip (electronic chip).

In an embodiment, the semiconductor chip may be configured for supporting or assisting the first sensor structure to electrochemically sense the physiological substance and/or the second sensor structure (104) to electrochemically sense the (physiological) parameter. For example, the semiconductor chip may be configured for detecting an electric voltage and/or an electric current. Thus, the semiconductor chip may replace or substitute a potentiostat for the electrochemical sensing.

In addition or alternatively, the semiconductor chip may be configured for transmitting sensed information of the first sensor structure and/or the second sensor structure to an evaluation unit of a monitoring system. By taking this measure, an immediate evaluation of the sensed information may be possible so as to immediately provide physiological results, which may further allow for an immediate reaction or response to the physiological results, such as therapeutic measures depending on the physiological results. In view of the high accuracy and reliability of the sensor arrangement, such immediate reaction to the physiological results may be performed by a patient himself without necessarily requiring a physician. In this regard, the evaluation unit may not only provide the physiological results, but also a proposal for a therapeutic regimen, for instance a proposal for an appropriate adjustment of medication depending on the physiological results.

Preferably, the semiconductor chip is configured for transmitting the sensed information in a wireless and/or contactless manner, in particular via near field communication (NFC). By taking this measure, sensed information of the first sensor structure and/or the second sensor structure to an evaluation unit of a monitoring system may be transmitted in a convenient and hygienic manner.

In an embodiment, the evaluation unit is (or forms) part of a portable electronic device, in particular a smartphone. Hereby, the results of the evaluation, such as the measured physiological results, may be directly displayed by the portable electronic device, for instance a smartphone. Moreover, the evaluation unit may not only provide the physiological results, but also a proposal for a therapeutic regimen, for instance a proposal for an appropriate adjustment of medication depending on the physiological results, such as an adjustment of diuretica medication in case of potassium sensing.

In an embodiment, the evaluation unit is configured for triggering an event depending on the sensed information indicative of hemolysis of the blood. For instance, the triggered event may be a refusal of the detection, a correction of a sensor result depending on sensed hemolysis, and outputting hemolysis information (in particular a corresponding alarm) to a user. Hence, in one embodiment, the patient may be informed (for instance by a display) that the measurement of the physiological substance must be rejected due to the occurrence of measurement disturbing hemolysis. The user may be invited to repeat the measurement. Additionally or alternatively, an algorithm may be carried out correcting the measured value of the physiological substance based on a determined degree of hemolysis. For instance, a lookup table may be used for this purpose from which correction parameters may be retrieved or a (correct, actual) value of the physiological substance may be calculated based on a determined degree of hemolysis. In yet another embodiment, the measurement result may be output to the user but may be accompanied with hemolysis information. For instance, an indication may be given to a user describing the reliability of the measurement results in view of the present hemolysis level. In still other embodiments, a visual, audible and/or haptic alarm may be generated as a warning to a user that the determined value of the physiological substance might lack reliability in view of detected hemolysis.

In an embodiment, the monitoring system comprises an accommodation recess shaped and dimensioned for accommodating at least part of the sensor arrangement so that the sensed information is transmittable from the sensor arrangement to the evaluation unit merely upon inserting the sensor arrangement into the accommodation recess. By such an intuitive insertion operation, a data communication connection may be established between (for instance sensor electrodes of) the test strip type sensor arrangement and (in particular the evaluation unit of) a base station comprising the accommodation recess. The evaluation unit may then process the sensed data to determine a result of the blood test.

In an embodiment, the sensor arrangement and/or the monitoring system may be suitably used in a therapeutic treatment of a person or animal in need thereof. Thus, a (therapeutic) method of treating a person or animal in need of a therapeutic treatment may involve a therapeutic measure (such as an adjustment of medication) depending on physiological results obtained/received from the sensor arrangement and/or the monitoring system as described herein.

The aspects defined above and further aspects of the invention are apparent from the examples of embodiment to be described hereinafter and are explained with reference to these examples of embodiment.

The invention will be described in more detail hereinafter with reference to examples of embodiment but to which the invention is not limited.

### BRIEF DESCRIPTION OF THE DRAWINGS

FIG. 1 shows a monitoring system with a sensor arrangement for monitoring a physiological substance according to an exemplary embodiment of the invention.

FIG. 2 shows a detailed view of the sensor arrangement of FIG. 1.

FIG. 3 shows an exploded view of components of a test strip type sensor arrangement for monitoring a physiological substance in blood of a physiological subject according to an exemplary embodiment of the invention.

FIG. 4 shows an assembled view of the sensor arrangement of FIG. 3.

FIG. 5 shows an assembled view of a sensor arrangement according to an exemplary embodiment of the invention illustrating, in addition to FIG. 4, also a second sensor structure and corresponding electrodes.

FIG. 6 shows results of the determination of hemolysis by means of a sensor arrangement according to an exemplary embodiment of the invention at various concentrations of hemoglobin.

### DETAILED DESCRIPTION OF ILLUSTRATED EMBODIMENTS

The illustrations in the drawings are schematically presented. In different drawings, similar or identical elements are provided with the same reference signs.

Before describing further exemplary embodiments in further detail, some basic considerations of the present inventors will be summarized based on which exemplary embodiments have been developed.

According to an exemplary embodiment of the invention, a potassium measurement and a hemolysis measurement may be combined in one common test strip. Various measurement techniques may be implemented for manufacturing such a sensor arrangement, including electrochemical elements, optical elements, etc. A corresponding test tool for capillary blood can be used in a self-test application in which a patient himself carries out the combined potassium and hemolysis test. A passive plasma separation can be carried out on a microfluidic test strip prior to the determination of a parameter and/or a physiological substance. Such a sensor arrangement is advantageous for the measurement of potassium and many other parameters (for instance measurement of ammonium, phosphate, lactate dehydrogenase (LDH), phosphatase, triglycerides, creatinine, and bilirubin) which can be measured highly accurately on the basis of plasma.

According to a more specific exemplary embodiment of the invention, a potassium test strip for a patient self-test is provided. Such a test strip is appropriate for carrying out patient self-test concerning potassium ion concentration in a capillary blood sample under consideration of possible artifacts due to hemolysis.

In an exemplary embodiment, a potassium sensor (which may be denoted more generally as first sensor structure) can be applied on a substrate (for instance made of plastic). Such a sensor may be composed of an ion selective electrode (ISE) and an Ag/AgCl reference electrode. Such a sensor arrangement can be manufactured completely by screen printing technique. In particular, screen printing is a highly appropriate manufacturing process for a corresponding electrode structure, a potassium sensitive membrane and a chloride reservoir membrane for the reference electrode. The potassium measurement can be carried out by the patient himself based on a drop of capillary blood from the finger pad. Such a measurement based on a drop of whole blood from the finger pad is conventionally prone for hemolysis (descriptively speaking a non-physiological modification of the blood caused by exerting excessive pressure onto the finger by a patient, i.e. by excessive squeezing). Since hemolysis can distort the measurement of the potassium concentration in the blood, an exemplary embodiment of the invention provides an additional hemolysis sensor (which may be denoted more generally as second sensor structure) on the test strip or sensor arrangement. In an embodiment, hemolysis detection is based on the detection of the redox current of iron in free hemoglobin at a sensor electrode. A passive filter system allows to separate the erythrocytes from the blood sample, since the erythrocytes and other cell constituents of the blood may disturb the hemolysis detection. Hemolysis detection is based, in an embodiment, on the determination of free hemoglobin in blood plasma. The sensor arrangement or test strip may advantageously further comprise a filter (which may be denoted more generally as plasma separation unit) for separating erythrocytes from the blood sample. Further advantageously, an exemplary embodiment of the invention may further comprise a microfluidic channel (which may be denoted more generally as fluidic channel structure) to guide the separated plasma along the sensor structure(s) in a passive and hemolysis-free way. The result of the hemolysis detection may be used to trigger a predefined event, such as the issuance of a warning to the patient that or when the potassium value is falsified by the occurrence of hemolysis. The result of the hemolysis detection may be further used to correct the determined value of the physiological substance so as to obtain an (at least more) accurate result of the actual value of the physiological substance (i.e. of the value of physiological substance without hemolysis).

Highly advantageously, the described additional hemolysis detection increases the safety during blood potassium determination by a patient and thereby enables the reliable and secure as well as precise self-measurement by a patient on the basis of capillary blood. Conventional test systems without hemolysis detection are allowed only for samples for venous whole blood samples, wherein a blood draw requires the involvement of health personnel.

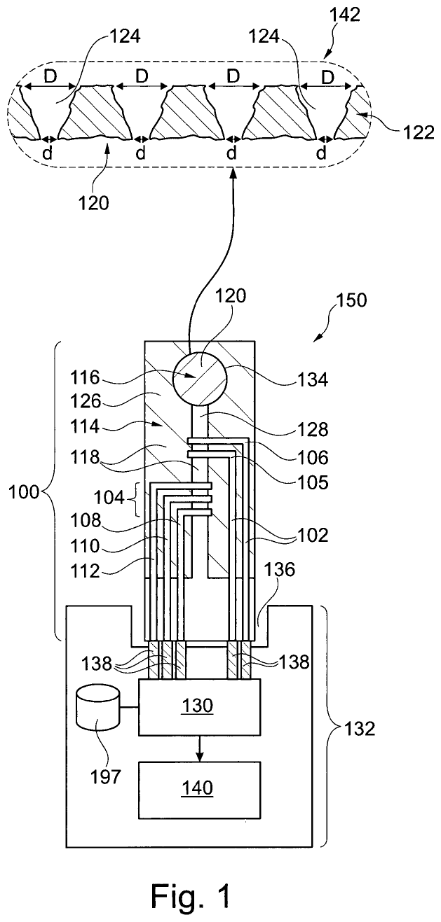

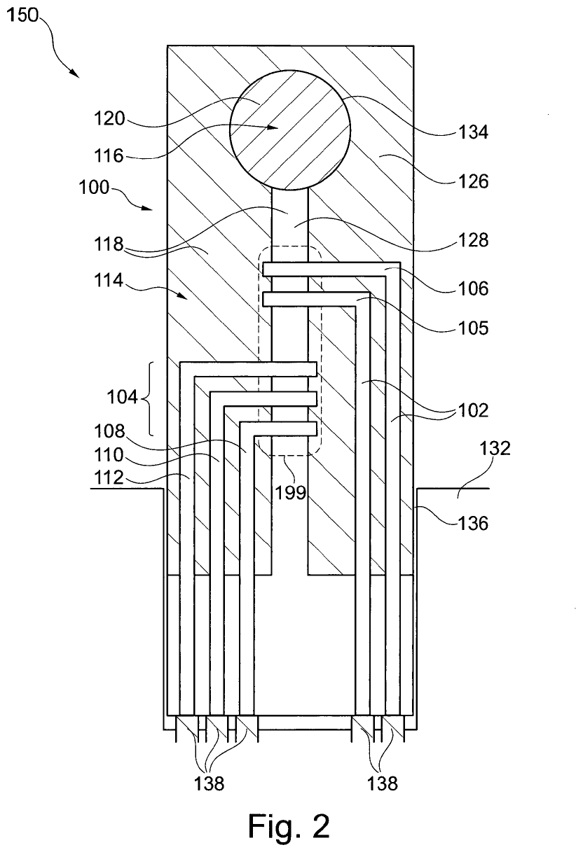

FIG. 1 shows a monitoring system 150 with a sensor arrangement 100 shaped as a flat sheet and configured for monitoring a physiological substance (in the shown embodiment a potassium ion level) in blood of a physiological subject (in the shown embodiment a human being) according to an exemplary embodiment of the invention. FIG. 2 shows a detailed view of the sensor arrangement 100 of FIG. 1.

The illustrated monitoring system 150 is composed of the test strip 114 type sensor arrangement 100 and a base station 132 comprising, in turn, an evaluation unit 130 for evaluating sensed information. The mentioned test strip 114 may be a substantially rectangular flat sheet for single use by a patient. More specifically, the sensor arrangement 100 can be embodied as a single-use disposable test strip 114 onto which a patient can place a self-drawn whole blood drop at a blood provision area 134. For the purpose of drawing a drop of whole blood, the patient can puncture his finger at the finger pad, for example using a needle (not shown). For instance, blood sampling may occur by finger pricking. The user may then place this finger on the blood provision area 134 of the sensor arrangement 100 and may apply a slight pressure on the finger pad to guide the drop of whole blood onto the blood provision area 134. Care should be taken to avoid overpressure during this procedure, since this may result in undesired hemolysis of the blood drop. However, when a medical layman, i.e. a patient himself, draws the blood, hemolysis may never be fully excluded in each and every case. Although the event of hemolysis may conventionally disturb the potassium measurement, a provision is taken in the sensor arrangement 100 to avoid false measurement results resulting from hemolysis. This will be described below in further detail.

The electronic base station 132 may be configured as a multiple use medical device (for instance a handheld device) having an accommodation recess 136 shaped and dimensioned for receiving an end portion of the test strip 114 like sensor arrangement 100 before or after the user has drawn a blood drop from his finger and has applied the same to the blood provision area 134. By inserting the sensor arrangement 100 into the accommodation recess 136 of the base station 132 of the monitoring system 150, an electrically conductive connection between electrodes 105, 106, 108, 110, 112 of the sensor arrangement 100 and counter electrodes 138 of the base station 132 may be established. As a result, electric potentials present at the electrodes 105, 106 being indicative of the potassium level and the current flow between electrodes 108 and 110 being indicative of the occurrence of hemolysis in the drawn blood drop may be supplied to the evaluation unit 134 for evaluation. For example, the evaluation unit 130 may be a processor (for instance a microprocessor or a central processing unit (CPU)) configured for executing machine executable code (for instance a software algorithm) for determining a level of the physiological substance (in the present embodiment the potassium ion level) in the drawn body fluid (in the shown embodiment blood). The evaluation unit 130 may be further configured for determining potential hemolysis in the same blood drop used also for determining the level of the physiological substance.

A result of this analysis may be output via a user interface 140 to a user. The user interface 140 may for example comprise an electronic display for displaying information to a user. Additionally or alternatively, the user interface 140 may also be configured for generating an acoustic output and/or a haptic output, for instance for indicating an alarm (such as a determined degree of hemolysis which renders the determined level of the physiological substance unreliable).

As indicated in FIG. 1, a plasma separation unit 116 is provided at the blood provision area 134. The plasma separation unit 116 is configured for separating the supplied whole blood sample into plasma on the one hand and cells on the other hand. Only the separated plasma portion is supplied to the below described sensor structures 102, 104, since the latter are only capable of reliably and precisely determining the respective potassium value and hemolysis level on the basis of plasma rather than whole blood. It is believed that the cells of the blood sample disturb such a measurement. The plasma separation unit 116 is hence configured for separating plasma from the blood and arranged so that only (or substantially only) the separated plasma is supplied to the first sensor structure 102 and the second sensor structure 104.

As can be taken from a detail 142 shown in FIG. 1, the plasma separation unit 116 may be embodied as a sheet like filter 120. In the shown embodiment, the sheet like filter 120 has a substantially circular shape so that a patient can put his finger pad directly onto the circular filter 120. As can be taken from the detail 142 showing a schematic cross-section of the filter 120 along a cross sectional plane perpendicular to the paper plane of FIG. 1, the filter 120 is formed by a permeable membrane 122 having pores 124. The pores 124 have a smaller diameter, d, on a main surface facing the sensor structures 102, 104 compared to a larger diameter, D, on an opposing other main surface facing a user supplying blood to the sensor arrangement 100. The relatively large pore size on the outer surface of the membrane 122 prevents large blood cells from traversing the membrane 122 and from clogging the pores 124. At the same time, blood plasma (including the small potassium ions to be sensed) is capable of traversing the membrane 122 through the pores 124. The smaller pore size on the interior surface of the membrane 122 facing the sensor structures 102, 104 ensures a proper permeability of the membrane 122 and therefore a rapid and precise detection. In other words, the configuration of the filter 120 ensures quick and substantially complete separation of plasma against cellular components of the blood sample while preventing undesired membrane clogging. Alternatively to the configuration of FIG. 1, it is however also possible to implement a filter membrane with homogeneous or isotropic pore dimensions.

As can be taken from FIG. 1 as well, the sensor arrangement 100 further comprises a microfluidic channel structure 118 configured for guiding specifically the separated plasma of the drawn whole blood sample to the first sensor structure 102 and the second sensor structure 104 exclusively by capillary action. More specifically, the microfluidic channel structure 118 comprises a flat sheet 126 with a covered recess forming a microfluidic channel 128 configured for guiding exclusively the separated plasma to the sensor structures 102, 104 by capillary action. Thus, the separated plasma is sucked into and along the microfluidic channel 128 without providing a drive mechanism such as a fluidic pump or the like, merely by the impact of capillary forces. This keeps the sensor arrangement 100 compact in size and simple in manufacture.

As can be taken from FIG. 1 and FIG. 2, the electrodes 105, 106, 108, 110, 112 of the sensor structures 102, 104 partially extend into the microfluidic channel 128 of the microfluidic channel structure 118, so that the separated plasma gets into physically contact with these electrodes 105, 106, 108, 110, 112. In other words, the electrodes 105, 106, 108, 110, 112 cross the microfluidic channel 128 in a crossing region 199 (see FIG. 2) so that the electrodes 105, 106, 108, 110, 112 detect electric signals as a result of an electrochemical interaction between the separated blood plasma and the electrodes 105, 106, 108, 110, 112. The electrodes 105, 106, 108, 110, 112 will thus detect electric signals allowing to determine both potassium ion concentration and possible hemolysis in the blood. As a consequence of the described design, the separated plasma may be reliably and precisely conducted to the electrodes 105, 106, 108, 110, 112 for detection without involving the risk of additional hemolysis on the fluidic path from the plasma separation unit 116 to the sensor structures 102, 104.

As an alternative to the microfluidic channel structure 118 designed according to FIG. 1, it is possible to provide a sheet of blotting paper in fluid communication with the electrodes 105, 106, 108, 110, 112. The separated plasma may then be soaked by the blotting paper and can thereby be brought in physical contact with the electrodes 105, 106, 108, 110, 112.

For carrying out the actual detection task in terms of potassium ion detection, the sensor arrangement 100 comprises the first sensor structure 102 configured for sensing the physiological substance (potassium in the described embodiment) in the previously separated plasma portion of the blood. Thus, the separated plasma separated by the plasma separation unit 116 is supplied to the sensor structures 102, 104 via the fluidic channel structure 118. In order to measure the potassium ion level electrochemically, the first sensor structure 102 comprises a reference electrode 105 (which is preferably embodied as a silver/silver chloride (Ag/AgCl) reference electrode, and a potassium ion-sensitive electrode 106. The electric voltage between electrodes 105, 106 which is generated in the presence of plasma of the drawn blood drop at the electrodes 105, 106 is a measure for the potassium level in the drawn blood sample. Since the phenomenon of hemolysis may change or influence the potassium concentration in the blood (upon drawing the blood sample), potassium sensing is undesirably influenced by hemolysis of the blood or in other words is hemolysis sensitive. This has the consequence that the measurement of the potassium level in the blood sample of the patient may be falsified when the phenomenon of hemolysis has occurred upon drawing the blood sample by the patient.

In order to overcome the danger of an undesired falsification of the potassium measurement signal in view of the presence of hemolysis, the second sensor structure 104 is provided on the test strip 114 type sensor arrangement 100 and is configured for sensing a hemolysis related parameter in the plasma portion of the drawn blood sample. More specifically, the mentioned additionally measured parameter is indicative of the possible event of hemolysis of the drawn blood sample. The second sensor structure 104 is configured for electrochemically sensing hemolysis in the drawn blood sample. For this purpose, the second sensor structure 104 comprises a reference electrode 108, a detection electrode 110, and a counter electrode 112.

The second sensor structure 104 may be configured for sensing hemolysis either qualitatively (i.e. hemolysis present or not) or quantitatively (i.e. detecting a level or an amount of hemolysis). When detecting hemolysis qualitatively, the presence of hemolysis may be assumed when a detected signal indicative of hemolysis exceeds a predefined threshold value. If the detected signal indicative of hemolysis remains below the predefined threshold value, it can be assumed that no hemolysis (or at least substantially no hemolysis) is present. The mere output of a qualitative indication of the presence (or absence) of hemolysis may be advantageous when a detected level of the physiological substance (such as potassium) is output to the user accompanied by the additional information as to whether the detected signal is reliable in view of the absence of hemolysis or is doubtful as a result of the presence of hemolysis. Such a yes/no logic is a simple guidance for a user as to whether the measurement of the physiological substance is reliable or should be repeated. When detecting hemolysis quantitatively, a gradual value may be output which is indicative of the signal amplitude measured for determining hemolysis. Alternatively, a corresponding one of multiple different hemolysis levels (for example no hemolysis, low hemolysis, moderate hemolysis, high hemolysis, etc.) may be output to the user. The quantitative estimation of hemolysis may also allow to carry out an electronic correction of the measured value of the physiological substance in view of the present intensity of hemolysis.

Highly advantageously, the first sensor structure 102 and the second sensor structure 104 are integrated in common test strip 114 in the embodiment of FIG. 1. As a result, both sensor structures 102, 104 are spatially very close together and get into physically contact with substantially the same blood sample. Consequently, the detection results of both sensor structures 102, 104 are directly comparable to one another and allow a precise consideration of potential hemolysis when interpreting the detected value of the physiological substance, in particular the potassium ion concentration.

As mentioned above, the evaluation unit 130 determines both the level of the physiological substance as well as possible hemolysis based on the sensor signals detected by the electrodes 105, 106, 108, 110, 112. The evaluation unit 130 is further configured for triggering an event depending on the sensed information indicative of hemolysis of the blood. Such a triggered event may be a refusal of the detection, the calculation of a correction of a sensor result depending on sensed hemolysis, and/or the output of hemolysis information to a user. In particular, an optical, acoustical and/or haptic warning or alarm may be output to the user as an indication that hemolysis has been detected and the determined value of the physiological substance might therefore be unprecise or unreliable.

When calculating a correction of the determined value of the physiological substance in view of the detected degree of hemolysis, the evaluation unit 130 may for instance carry out the correction based on correction data which may be stored in a data storage unit 197 (such as a mass storage device, for instance a hard disk) of the base station 132. The evaluation unit 130 may access the data storage unit 197 for reading and/or writing data. For instance, a correction value may be taken from a lookup table (stored in data storage unit 197) correlating a respective amount of hemolysis with a correction parameter by which the determined value of the physiological substance has to be multiplied to obtain a true value of the physiological substance which would be measured in the absence of hemolysis. In a further refined embodiment, the correcting calculation may be performed on the basis of more than one correction parameter. The correction values or parameters may be determined by carrying out reference measurements of the physiological substance at different levels of hemolysis, may be determined on the basis of a theoretical model of hemolysis and its influence of the measurement of the physiological substance, and/or may be determined based on expert knowledge (such as expert rules).

Concluding, FIG. 1 and FIG. 2 show test strip 114 with a potassium and hemolysis sensing capability. The filter 120 ensures a hemolysis free separation of erythrocytes from the drawn blood sample and ensures that the potassium and hemolysis detection are carried out on the basis of pure plasma. Advantageously, the tiny microfluidic channel 128 transports selectively the separated plasma to the mutually closely arranged electrodes 105, 106, 108, 110, 112 of the sensor structures 102, 104 for electrochemical detection of potassium and hemolysis based on the same sample.

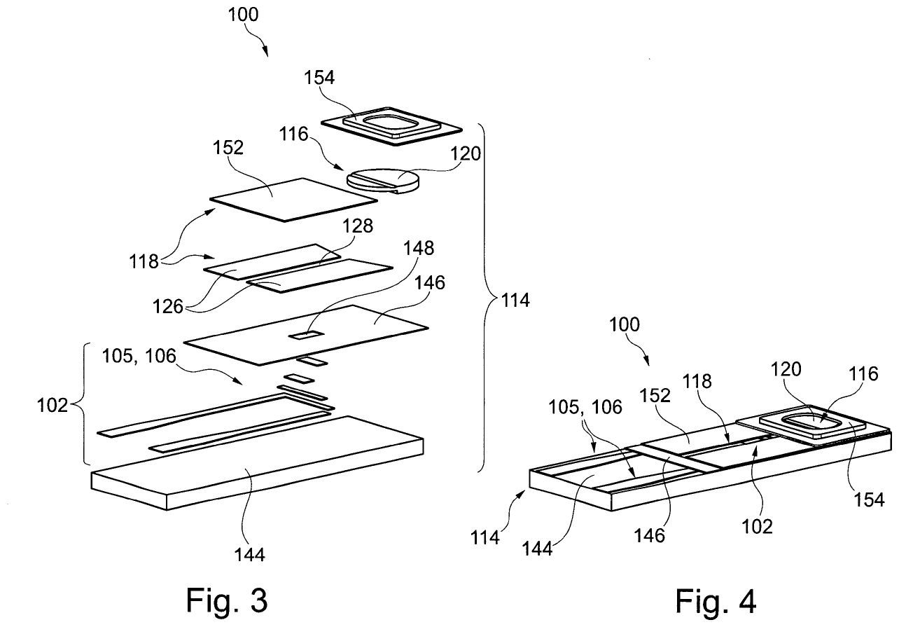

FIG. 3 shows an exploded view of various components of a test strip 114 type sensor arrangement 100 for monitoring a physiological substance in a physiological subject according to an exemplary embodiment of the invention. FIG. 4 shows an assembled view of the sensor arrangement 100 of FIG. 3. The second sensor structure 104 and corresponding electrodes 108, 110, 112 are not illustrated in FIG. 3 and FIG. 4.

As can be taken from FIG. 3 and FIG. 4, the first sensor structure 102, the plasma separation unit 116, the fluidic channel structure 118, and consequently the sensor arrangement 100 as a whole are manufactured on the basis of stacked flat sheet structures. More specifically, all the mentioned constituents are arranged on a common flat substrate 144. The substrate 144 may be a rectangular sheet of plastic or the like and serves as a support or mounting base for the other constituents of the sensor arrangement 100. The various planar electrodes 105, 106 of the first sensor structure 102 are formed on or attached to the substrate 144. Thereafter, a cover layer 146 with a central through hole 148 may be attached on the substrate 144 covered with the electrodes 105, 106. Specific portions of the electrodes 105, 106 are exposed in an upward direction by the through hole 148. Subsequently, the sheet type microfluidic structure 118 is attached on top of the cover layer 146. More specifically, the sheet 126 is put on the cover layer 146 so that the microfluidic channel 128 is in fluid communication with the through hole 148, and therefore with the exposed portions of the electrodes 105, 106. Subsequently, a lid layer 152 is put on top of the sheet 126 to cover the microfluidic channel 128, thereby delimiting a circumferentially closed microfluidic capillary. The circular filter 120 of the plasma separation unit 116 is assembled so as to be in fluid communication with the microfluidic channel 128. A filter fixation unit 154, which is here embodied as an annular sheet, is put onto the filter 120 for mechanically fixing the latter.

The constituents of the test strip 114 according to FIG. 3 and FIG. 4 may be manufactured by screen printing.

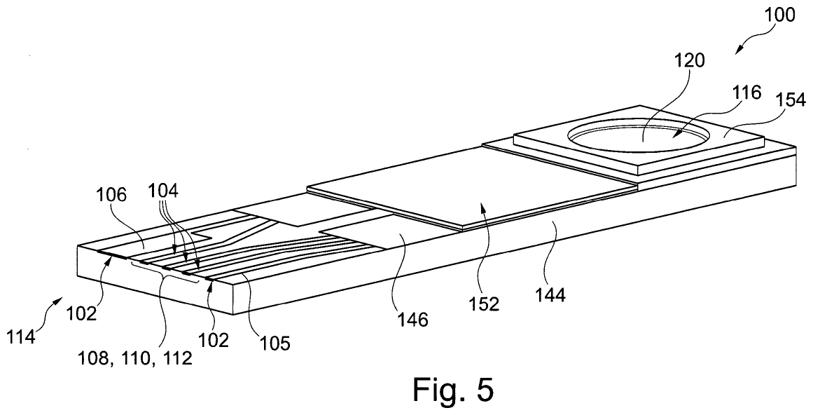

FIG. 5 shows an assembled view of a sensor arrangement 100 according to an exemplary embodiment of the invention illustrating, in addition to the components shown in FIG. 3 and FIG. 4, also a second sensor structure 104 and corresponding electrodes 108, 110, 112. Also, the planar electrodes 108, 110, 112 of the second sensor structure 104 of the test strip 114 according to FIG. 5 can be embodied as flat sheets. According to FIG. 5, the fluidic channel structure 118 is arranged (for example as a groove) within lid layer 152 and is therefore not visible in FIG. 5.

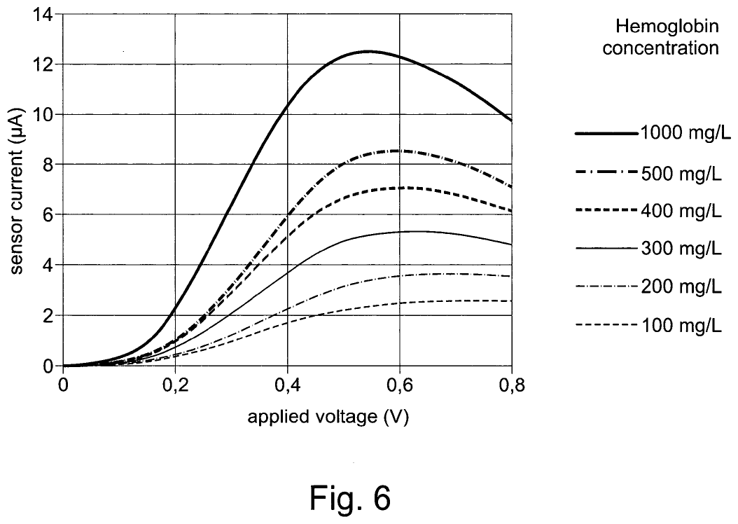

FIG. 6 shows results of the determination of hemolysis by means of a sensor arrangement according to an exemplary embodiment of the invention at various concentrations of hemoglobin. More specifically, hemolysis is measured by direct oxidation of hemoglobin in plasma at the working electrode of the sensor. The sensor signal, measured as sensor current, reaches a maximum at a working potential of 500 mV for a certain hemoglobin concentration. The measured sensor current maximum at 500 mV correlates with the hemoglobin concentration in the sample as depicted in FIG. 6, and can thus be used to indicate hemolysis in the sample. As it is further evident from FIG. 6, the sensor arrangement according to an exemplary embodiment of the invention may operate well over a broad concentration range and may thus be used in a versatile manner for correcting the results of a sensed hemolysis-sensitive physiological substance.

It should be noted that the term “comprising” does not exclude other elements or steps and the article “a” or “an” does not exclude a plurality. Also, elements described in association with different embodiments may be combined.

Implementation of the invention is not limited to the preferred embodiments shown in the figures and described above. Instead, a multiplicity of variants is possible which use the solutions shown and the principle according to the invention even in the case of fundamentally different embodiments.

1. A sensor arrangement for detecting information indicative of a physiological substance in blood of a physiological subject, the sensor arrangement comprising:
a first sensor structure configured for electrochemically sensing the physiological substance in at least part of the blood, wherein detecting the physiological substance is influenceable by hemolysis of the blood;
a second sensor structure configured for electrochemically sensing a parameter in at least part of the blood, which parameter is indicative of hemolysis of the blood; and
a plasma separation unit configured for separating plasma from the blood and arranged so that the separated plasma is supplied to the first sensor structure and the second sensor structure, whereas a rest of the blood is kept apart from the first sensor structure and the second sensor structure.

2. The sensor arrangement according to claim 1, wherein the first sensor structure is configured for sensing potassium as the physiological substance.

3. The sensor arrangement according to claim 1, wherein the first sensor structure comprises a reference electrode, in particular a silver/silver chloride reference electrode, and an ion-sensitive electrode.

4. The sensor arrangement according to claim 1, wherein the second sensor structure comprises a reference electrode, a detection electrode and a counter electrode.

5. The sensor arrangement according to claim 1, wherein the first sensor structure and the second sensor structure are arranged on and/or integrated in a common test strip, in particular as a disposable test strip operable by a patient.

6. The sensor arrangement according to claim 1, wherein the second sensor structure is configured for sensing hemolysis qualitatively or quantitatively.

7. The sensor arrangement according to claim 1, wherein the plasma separation unit comprises or consists of a filter, in particular a permeable membrane, more particularly a permeable membrane having pores which have smaller diameters on a main surface facing the sensor structure compared to larger diameters on an opposing other main surface facing a user supplying blood to the sensor arrangement.

8. The sensor arrangement according to claim 1, comprising a fluidic channel structure, in particular a microfluidic channel structure, configured for guiding at least part of the blood to the first sensor structure and the second sensor structure by capillary action.

9. The sensor arrangement according to claim 1, wherein at least one of the group consisting of the first sensor structure, the second sensor structure, the plasma separation unit, the fluidic channel structure, and the sensor arrangement as a whole, is configured as a flat sheet.

10. The sensor arrangement according to claim 1, comprising a semiconductor chip, in particular a semiconductor chip configured for transmitting sensed information of the first sensor structure and/or the second sensor structure to an evaluation unit of a monitoring system.

11. The sensor arrangement according to claim 10, wherein the semiconductor chip is configured for transmitting the sensed information in a wireless and/or contactless manner, in particular via near field communication.

12. A monitoring system, comprising:
a sensor arrangement having a first sensor structure, a second sensor structure and a plasma separation unit, the first sensor structure configured for electrochemically sensing the physiological substance in at least part of the blood, wherein detecting the physiological substance is influenceable by hemolysis of the blood, the second sensor structure configured for electrochemically sensing a parameter in at least part of the blood, which parameter is indicative of hemolysis of the blood, the plasma separation unit configured for separating plasma from the blood and arranged so that the separated plasma is supplied to the first sensor structure and the second sensor structure, whereas a rest of the blood is kept apart from the first sensor structure and the second sensor structure; and
an evaluation unit configured for evaluating sensed information.

13. The monitoring system according to claim 12, wherein the evaluation unit is part of a portable electronic device, in particular a smartphone.

14. The monitoring system according to claim 12, wherein the evaluation unit is configured for triggering an event depending on sensed information indicative of hemolysis of the blood.

15. The monitoring system according to claim 14, wherein the triggered event comprises at least one of the group consisting of a refusal of the blood test, a correction of a sensor result depending on sensed hemolysis, and an output of hemolysis information, in particular an alarm, to a user.

16. The monitoring system according to claim 12, comprising an accommodation recess shaped and dimensioned for accommodating at least part of the sensor arrangement so that the sensed information is transmittable from the sensor arrangement to the evaluation unit upon inserting the sensor arrangement into the accommodation recess.

17. A method of detecting information indicative of a physiological substance in blood of a physiological subject, the method comprising:
separating plasma from the blood;
electrochemically sensing the physiological substance in at least part, in particular the separated plasma, of the blood, wherein detecting the physiological substance is influenced by hemolysis of the blood;
electrochemically sensing a parameter in at least part, in particular the separated plasma, of the blood, which parameter is indicative of hemolysis of the blood.
